(19) United States
(12) Patent Application Publication
(10) Pub. No.: US 2005/0059166 A1
(43) Pub. Date: Mar. 17, 2005

(54) Sampling instrument

(76) Inventor: Robert Markes, Omer (IL)

Correspondence Address: Dekel Patent Ltd.;Beit HaRofim, Room 27, 18 Menuha VeNahala Street, Rehovot, IL

(21) Appl. No.: 10/659,291

(22) Filed: Sep. 11, 2003

(51) Int. Cl. C12M 1/34; G01N 33/558

(52) U.S. Cl. 436/514; 435/287.2; 604/500

(57) ABSTRACT

A biological sampling/measuring instrument including a first housing including a needle arranged for protrusion therefrom, the needle being adapted to draw therethrough a biological fluid, a reagent disposed in the first housing in fluid communication with the needle, capable of producing an optically-sensible reaction with a biological fluid, and an optical sensor disposed in the first housing adapted to sense the optically-sensible reaction. In one embodiment, the instrument is an elongate instrument suitable for taking and testing small blood samples from a human. The instrument may have a disposable part and a reusable part to which the disposable part connects.

### FIELD OF THE INVENTION

This invention relates to sensors for use in biological, microbiological, biochemical and chemical testing, and particularly to instruments and/or sensors used to monitor the presence of a substance, such as but not limited to, monitoring the interaction of antibodies with their corresponding antigens and for detecting the presence of antigens, for example.

### BACKGROUND OF THE INVENTION

Devices are known for optically detecting substances. For example, U.S. Pat. No. 6,346,376 to Sigrist, et al. describes an optical sensor unit for the specific detection and identification of biomolecules at high sensitivity in real fluids and tissue homogenates. High detection limits are reached by the combination of i) label-free integrated optical detection of molecular interactions, ii) the use of specific bioconstituents for sensitive detection and iii) planar optical transducer surfaces appropriately engineered for suppression of non-specific binding, internal referencing and calibration

Another example of another kind of optical sensor device is U.S. Pat. No. 5,859,937 to Nomura, which describes a minimally invasive sensing device, which utilizes a light-conducting fiber having a localized textured site thereon, wherein a reagent is deposited. Interaction of the reagent with an analyte specific to the reagent produces a response, such as development of a colored product, which is detectable by means of a change in characteristics of a light beam transmittable through the fiber. The sensor may be useful in blood glucose determinations, requiring smaller blood samples than flat strip devices.

Still other optical devices are known that operate on other optical principles. For example, when antibodies are immobilized on a surface, the properties of the surface change when a solution containing a corresponding antigen is brought into contact with the surface to thus allow the antigen to bind with the antibody. In particular, the change in the optical properties of the surface can be monitored with suitable apparatus.

The phenomenon of surface plasmon resonance (SPR) can be used to detect minute changes in the refractive index of the surface as the reaction between the antigen and the antibody proceeds. Surface plasmon resonance is the oscillation of the plasma of free electrons which exists at a metal boundary. These oscillations are affected by the refractive index of the material adjacent the metal surface and it is this that forms the basis of the sensor mechanism. Surface plasmon resonance may be achieved by using the evanescent wave which is generated when a p-polarized light beam is totally internally reflected at the boundary of a medium, e.g. glass, which has a high dielectric constant.

For example, U.S. Pat. No. 5,573,956 to Hanning describes a method of assaying for an analyte in a fluid sample comprises detecting the presence of the analyte by determining the resulting change in refractive index at a solid optical surface in contact with the sample, which change is caused by the analyte involving or influencing the binding or release of a refractive index-enhancing species to or from, respectively, the optical surface. Determination is performed with light having a wavelength at or near the maximum of the negative derivative of the absorptivity with respect to wavelength of the refractive index-enhancing species to obtain maximum sensitivity.

### SUMMARY OF THE INVENTION

The present invention seeks to provide a novel apparatus for detecting the presence of a substance, such as but not limited to, monitoring the interaction of antibodies with their corresponding antigens and for detecting the presence of antigens, as is described more in detail hereinbelow.

There is thus provided in accordance with an embodiment of the invention a biological sampling instrument including a first housing including a needle arranged for protrusion therefrom, the needle being adapted to draw therethrough a biological or environmental fluid, a reagent disposed in the first housing in fluid communication with the needle, capable of producing an optically-sensible reaction with a biological fluid, and an optical sensor disposed in the first housing adapted to sense the optically-sensible reaction.

The term “needle” encompasses any device for drawing fluid therethrough, such as but not limited to, a needle, capillary tube, aspiration device, suction device, fluid conduit and the like. The term “sampling” encompasses sampling, measuring, analyzing, processing and the like.

In accordance with an embodiment of the invention a processor in communication with the optical sensor adapted to process a signal from the optical sensor, the signal being a function of the optically-sensible reaction. The processor may be in communication with the optical sensor by means of an optical waveguide.

Further in accordance with an embodiment of the invention the processor may be disposed in a second housing, the first and second housings including mating connectors to effect the communication between the processor and the optical sensor. The first housing may be disposable. The second housing may be reusable. The first and second housings together may form an elongate housing

Still further in accordance with an embodiment of the invention a fluid pump may be in fluid communication with the needle, adapted to pump a biological fluid through the needle.

In accordance with an embodiment of the invention the first housing further includes a waste receptacle for storing therein waste products of the optically-sensible reaction. Further in accordance with an embodiment of the invention a destructive fluid is available at the end to neutralize the fluids introduced such as HIV, hepatitis or other.

Further in accordance with an embodiment of the invention the needle may be retractable into the first housing.

Still further in accordance with an embodiment of the invention a display may be in communication with the processor. The processor may include a photodiode and a microprocessor, for example.

### BRIEF DESCRIPTION OF THE DRAWINGS

The present invention will be understood and appreciated more fully from the following detailed description taken in conjunction with the drawings in which:

FIG. 1 is a simplified pictorial illustration of a biological sampling instrument, constructed and operative in accordance with an embodiment of the present invention, showing first and second housings separate from each other;

FIG. 2 is a simplified pictorial illustration of the sampling instrument of FIG. 1, showing the first and second housings connected to each other.

### DETAILED DESCRIPTION OF THE PREFERRED EMBODIMENT

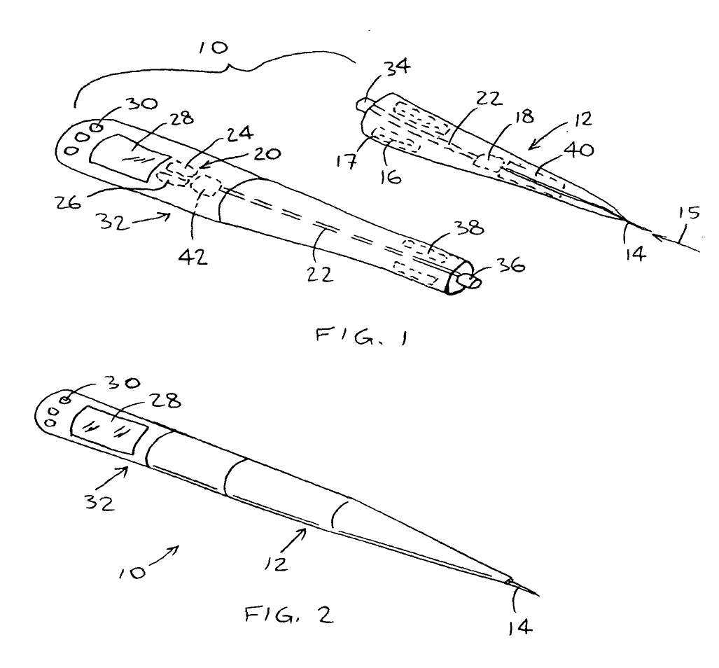

Reference is now made to FIGS. 1 and 2, which illustrate a biological sampling instrument 10, constructed and operative in accordance with an embodiment of the present invention.

Instrument 10 may comprise a first housing 12 comprising a needle 14 arranged for protrusion therefrom. Needle 14 is adapted to draw therethrough a biological fluid 15, such as but not limited to, blood, lymph, saliva, sweat and the like. Needle 14 may be retractable into first housing 12. One or more reagents 16 may be disposed in one or more compartments 17 first housing 12 in fluid communication with needle 14. Reagent 16 may comprise any substance capable of producing an optically-sensible reaction with the biological fluid 15. An optical sensor 18 may be disposed in first housing 12 for sensing the optically-sensible reaction, as is described more below.

A processor 20 may be in communication with optical sensor 18, such as by means of one or more optical waveguides 22 (the term “optical waveguide” also encompasses an optical fiber). Processor 20 may process a signal from optical sensor 18, the signal being a function of the optically-sensible reaction. For example, processor 20 may comprise a microprocessor 24 and photodiode 26. Photodiode 26 may convert the light emission from optical waveguide 22 to a current and then to a display 28 in communication with processor 20. Any number and kind of control buttons 30 (e.g., on/off, display functions, command keys, etc.) may be provided.

Alternatively or additionally, a transmitter and/or receiver may be provided for wireless communication with an external processor, server, website, etc., such as but not limited to, radio transmission, cell phone transmission, infrared transmission and the like.

Processor 20 and display 28 may be disposed in a second housing 32. First and second housings 12 and 32 may together form an elongate housing. First and second housings 12 and 32 may comprise mating connectors 34 and 36, respectively, to effect the communication between processor 20 and optical sensor 18. First housing 12 may be disposable, whereas second housing 32 may be reusable.

Optionally fluid pump 38 may be in fluid communication with needle 14 to assist pumping and drawing the biological fluid 15 through needle 14. The fluid pump 38 may be disposed in second housing 32, but alternatively may be disposed in first housing 12.

First housing 12 may also comprise a waste receptacle 40 for storing therein waste products of the optically-sensible reaction. Waste receptacle 40 may alternatively or additionally be used for storing therein a destructive fluid for neutralizing fluids, such as but not limited to, HIV or hepatitis inflected fluids and the like.

In operation of biological sampling instrument 10, as mentioned above, optical sensor 18 may sense the optically-sensible reaction of reagent 16 with biological fluid 15. For example, interaction of reagent 16 with an analyte in the biological fluid 15 specific to reagent 16 may produce a colored product, which is detectable by optical sensor 18 means of a change in characteristics of a light beam transmitted thereat. As such, biological sampling instrument 10 may further comprise a light source 42 of coherent or non-coherent light. Light source 42 may transmit a light beam to the reaction site via optical waveguide 22. Alternatively, light source 42 may be disposed in first housing 12. It is appreciated that the invention is not limited to this example, and other optical phenomena may be detected by a suitable optical sensor 18, such as but not limited to, surface plasmon resonance (SPR).

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

1. A sampling instrument comprising:
a first housing comprising a needle arranged for protrusion therefrom, said needle being adapted to draw therethrough a fluid;
a reagent disposed in said first housing in fluid communication with said needle, capable of producing an optically-sensible reaction with a fluid; and
an optical sensor disposed in said first housing adapted to sense said optically-sensible reaction.

2. The sampling instrument according to claim 1, further comprising a processor in communication with said optical sensor adapted to process a signal from said optical sensor, said signal being a function of said optically-sensible reaction.

3. The sampling instrument according to claim 2, wherein said processor is in communication with said optical sensor by means of an optical waveguide.

4. The sampling instrument according to claim 2, wherein said processor is disposed in a second housing, said first and second housings comprising mating connectors to effect the communication between said processor and said optical sensor.

5. The sampling instrument according to claim 1, wherein said first housing is disposable.

6. The sampling instrument according to claim 2, further comprising a fluid pump in fluid communication with said needle adapted to pump a biological fluid through said needle.

7. The sampling instrument according to claim 4, further comprising a fluid pump in fluid communication with said needle adapted to pump a biological fluid through said needle, wherein said fluid pump is disposed in said second housing.

8. The sampling instrument according to claim 1, wherein said first housing further comprises a waste receptacle for storing therein at least one of waste products of said optically-sensible reaction and destructive fluids for neutralizing substances.

9. The sampling instrument according to claim 1, wherein said needle is retractable into said first housing.

10. The sampling instrument according to claim 2, further comprising a display in communication with said processor.

11. The sampling instrument according to claim 2, wherein said processor comprises a photodiode and a microprocessor.

12. The sampling instrument according to claim 1, further comprising at least one of a transmitter and a receiver for wireless communication with an external device.

13. The sampling instrument according to claim 4, wherein said first and second housings together form an elongate housing.

14. The sampling instrument according to claim 4, wherein said second housing is reusable.
